(19) United States
(12) Patent Application Publication
(10) Pub. No.: US 2012/0329163 A1
(43) Pub. Date: Dec. 27, 2012

(54) MULTI-WELL PLATE WITH FILTER MEDIUM, AND USE THEREOF

(75) Inventors: Rene Faber, Goettingen (DE); Martin Leuthold, Goettingen (DE)

(73) Assignee: SARTORIUS STEDIM BIOTECH GMBH, Goettingen (DE)

(21) Appl. No.: 13/575,396

(22) PCT Filed: Feb. 2, 2011
(86) PCT No.: PCT/EP2011/000468

§ 371 Date: Jul. 26, 2012

(30) Foreign Application Priority Data
Mar. 16, 2010 (DE) 10 2010 011 485.5

(51) Int. Cl. G01N 1/34; G01N 33/68; G01N 21/75

(52) U.S. Cl. 436/86; 436/164; 422/535

(57) ABSTRACT

A multi-well plate (1) has an upper part (2) with a multiplicity of wells (3), a lower part (4) with a multiplicity of wells (5) that communicate with the wells (3) of the upper part (2), and at least one filter medium (6) that can be fixed between the upper and lower parts (2, 4). Sides (8, 9) of the upper and lower parts (2, 4), facing toward the filter medium (6), have seals (7a, 7b) extending around the wells. The filter medium (6) can be fixed along the upper and lower sides in each case by pairs of the seals (7b) of the upper part (2) and the seals (7a) of the lower part (4). The multi-well plate prevents cross-contamination between adjacent wells due to radial cross-diffusion of analytes. A method for characterization of filter media using the multi-well plate (1) also is provided.

### BACKGROUND OF THE INVENTION

1. Field of the Invention

The present invention relates to a multi-well plate with an upper part and a lower part, each having a multiplicity of communicating wells, and with a filter medium, which can be fixed between the upper part and the lower part by sealing means, and to the use thereof for high throughput analyses for characterization of membranes for separation of substances by adsorption.

2. Description of the Related Art

For characterization of membranes for separation of substances by adsorption, many parameters have to be examined. Especially for optimization in the elaboration of biotechnology products, it is not practicable to carry out individual tests to examine the influence of media properties (nature and composition) or the interaction of proteins with other compounds. High throughput screening systems (HTS systems) or multi-well systems are increasingly being used, with which it is possible to test a large number of parameters of influence in a short time and with reduced consumption of expensive media. Such systems are already widely used in gel chromatography. Applying HTS techniques of this kind to membrane adsorbers as filter media is of great interest.

EP 1 566 209 A1 describes an affinity chromatography device for protein purification, which allows different filter media (e.g. membranes) to be used for test purposes. After centrifugation of the sample that is to be analyzed, and with application of a vacuum, proteins from large loading volumes can be bound to the filter medium and then concentrated by elution in a small volume. The microfilters used can also be combined with an ultrafilter. The device can be combined with collecting devices of different sizes. In a preferred embodiment, the membrane disk placed in the device is sealed off from one side by an 0-ring. Since only one medium can be filtered with one device, this device is unsuitable for parallel screening of a plurality of samples.

U.S. Pat. No. 4,427,415 describes a multi-well plate as vacuum filtration unit for simultaneous analysis of a plurality of substances. The individual wells of an upper part are equipped with individual filter paper disks, after which solutions with different reagents are successively introduced into the wells and filtered. The reagents are held back by the filter paper. The multi-well plate can also optionally be equipped with just one continuous sheet of filter paper that covers all the wells of the upper part. By successive filtration processes, different reagents held back by the filter can thus react with one another and can then be investigated (microscopy, spectrometry). The filtration unit is operated by vacuum and is placed on a vacuum unit by way of a plastic gasket, the seal being arranged peripherally in a depression of the vacuum unit. As far as undesired cross-contamination between adjacent wells is concerned, the non-optimized fluid discharge from the cylindrical wells, which is possible only with application of a vacuum, and the cross-diffusion on the filter paper are problematic, because the multi-well plate has no sealing means between upper part and lower part for ensuring individual sealing of the individual wells in the upper part.

WO 01/87484 A2 describes a disposable multi-well filter plate, in which a membrane filter sheet is affixed to the lower surface of the filter plate. The filter sheet consists of an upper and lower surface, wherein the upper surface has incisions passing partially through the filter thickness for penetration of an adhesive into the filter sheet, so as to form a seal and connect the filter sheet to the underside of the plate. The use of an adhesive as sealant is unacceptable for many biotechnology applications because of the possible contamination of the filtrate by dissolved components of the adhesive. Moreover, replacement of the filter sheet in the filter plate is not possible.

U.S. Pat. No. 4,902,481 discloses a multi-well filtration unit with an upper part and a lower part, in which the individual wells are isolated from one another in a fluid-tight manner and are sealed by pressing the upper part onto the lower part or by adhesion or snap-fit connection between upper part and lower part. Each individual well is equipped with an individual membrane disk. The outlets of the individual wells are so narrow that no flow is possible by gravity alone, and the drops of the filtrate that form and remain at the outlet can produce an undesired cross-mixing between adjacent wells.

For the purification of proteins, multi-well strips from Sartorius Stedim Biotech GmbH are known (“Vivawell Vac8 purification strips”) in which eight individual wells are equipped with individual membranes. Twelve of these strips can be placed in a holder to form a multi-well plate with 96 wells. In the strips, the membranes of the individual wells are completely separated from one another in a fluid-tight manner. However, a great disadvantage of this device is that each individual hole is equipped with an individual membrane cutout. These cutouts have to be cut individually, fitted into the wells and sealed off by sleeve inserts, which are inserted into the individual wells. Furthermore, a uniform flow through the membrane cutouts is not ensured, because the membrane cutouts lie partially on the well bottom on the permeate side. Similar disadvantages also arise for other commercially available plates (“Filter Bottom Microplates” from Seahorse Bioscience) for screening applications. These are offered in particular for ultrafiltration. The outflow and the sealing of the wells are not designed for membrane adsorbers. Here too, the wells have to be equipped individually.

U.S. Pat. No. 5,047,215 discloses a multi-well plate, of which the lower part has, around each opening (each well), an integrally formed and annular bead, which engages in a corresponding annular recess on the underside of the upper part of the multi-well plate, wherein a microporous membrane sheet is placed between upper part and lower part. The upper part is welded to the lower part to seal off the individual wells, such that separate membrane sections are present in the individual wells of the plate after the upper part has been welded to the lower part. Apart from the fact that the separated membrane sections cannot easily be removed from the multi-well plate for further analysis after the filtration, the aforementioned welding has an adverse effect on membrane materials with low thermal loading capacity, e.g. materials based on polysaccharides, such that the filtration properties of the membrane sections may be impaired in the edge areas of the membrane sections lying adjacent to the seal that has been produced by welding.

EP 1 854 542 A1 discloses a multi-well plate with an upper filter plate having a multiplicity of tubes which each enclose the upper cylindrical sections of the channels of the wells. The multi-well plate also has a lower part, which comprises the lower sections of the wells, which lower sections consist of a frustoconical transition area and, adjoining the latter, a cylindrical lower tube part whose diameter is smaller than the diameter of the cylindrical channel sections in the upper part. Individual filter disks are placed in each of the wells and come to lie on the upper edge of the frustoconical transition areas in the lower part.

U.S. Pat. No. 5,939,024 discloses a multi-well plate in which cross-contamination between the individual wells is minimized and which consists of an upper part (“collimator”), a lower part (“holding tray”) and, placed between these, a filter sheet. To ensure that cross-contamination caused by radial diffusion of analytes between the individual wells is reduced, the lower part of each well channel in the upper part has an annular bulge (“lower rim”) of semicircular cross section, by means of which the filter sheet is pressed across its whole surface area onto the plane surface of the lower part. The device is used in autoradiography and for liquid scintillation counting techniques, in which a filter sheet, on which analyte is immobilized, is placed between upper part and lower part, and the membrane segments in the individual wells are then subjected to treatments with further reagents.

This device has in principle proven to be excellent for the aforementioned methods but has the disadvantage that, because of the inadequate sealing of the individual wells and the plane construction of the support surface for the filter sheet in the lower part (“holding tray”), it is unsuitable for filtration processes which involve separation of substances by adsorption and in which (bio)chemical binding reactions have to be quantitatively balanced.

When a stack of several membrane adsorber sheets is used in a multi-well plate according to U.S. Pat. No. 5,939,024 for separation of substances by adsorption, undesired cross-contamination is observed between the individual wells as a result of radial cross-diffusion beyond the individual well, which is sealed off only by the annular bulges on the upper part. When a stack of several membrane adsorber sheets is used as filter medium, the sealing means proposed in U.S. Pat. No. 5,939,024 are not sufficient to exclude the possibility of cross-contamination between adjacent wells. This undesired spread of analytes beyond the peripheral well boundaries causes balancing errors in respect of the adsorption of the analyte per membrane surface, e.g. in binding studies.

### SUMMARY OF THE INVENTION

Therefore, the object of the present invention is to make available a multi-well plate in which, during high throughput analysis (high throughput screening), cross-contamination caused by cross-diffusion of the analyte beyond the well boundaries is excluded.

A further object of the present invention is to propose a use of the multi-well plate according to the invention for characterization of membranes for separation of substances by adsorption.

These objects are achieved by the embodiments of the present invention that are characterized in the claims.

The object concerning the multi-well plate is achieved by a multi-well plate that comprises
an upper part with a multiplicity of wells,
a lower part with a multiplicity of wells, which communicate with the wells of the upper part, and
at least one filter medium, which can be fixed between the upper part and the lower part,
   wherein the upper part and the lower part, on the sides thereof facing toward the filter medium, have sealing means extending around the wells, and the filter medium can be fixed along the upper side and lower side thereof in each case by pairs of the sealing means of the upper part and the sealing means of the lower part.

By virtue of the fixing of the filter medium by corresponding pairs of sealing means on upper part and lower part, cross-contamination is reliably prevented between adjacent wells, which cross-contamination would distort the substance balance for the section of the filter medium enclosed in a well.

According to one embodiment of the invention, the filter medium comprises a membrane sheet.

In another embodiment of the invention, the filter medium comprises a multiplicity of membrane sheets stacked one on top of another.

The device according to the invention has proven particularly useful when using filter media comprising a plurality of stacked membrane sheets, for example up to 10 membrane sheets, preferably up to six membrane sheets and most preferably up to four membrane sheets, for avoiding cross-contamination or cross-diffusion between adjacent wells.

In these two aforementioned embodiments, the membrane sheet, either as the sole component of the filter medium or as a constituent of a stack of membrane sheets forming the filter medium, is preferably a microporous membrane to which ligands are bound via optional spacers. The ligands can be functional groups that undergo chemical or physical interactions with the analytes that are to be examined. Chemical or physical interaction is understood as any type of ionogenic, covalent, polar or hydrophobic interaction between the ligands and the molecules of the analyte.

Particularly preferably, the membrane sheet consists of cellulose derivatives, particularly preferably cellulose or cellulose acetate.

In the aforementioned embodiments, the filter medium is preferably wetted with a fluid, i.e. the filter medium is preferably in a moist state, e.g. after complete wetting by a fluid (e.g. a buffer solution or saline solution), when fixed in the device according to the invention, before an analysis is carried out using the device according to the invention.

According to a preferred embodiment, the sealing means are annular sealing beads which are formed integrally on the sides of the lower part and upper part facing toward the filter medium.

It is alternatively possible to form depressions around the wells on the sides of the upper part and/or lower part facing toward the filter medium, into which depressions O-rings can be placed or fixed as sealing means.

Alternatively, the sealing means can be annular sealing beads which are milled out on the sides of the lower part and of the upper part facing toward the filter medium. This embodiment permits rapid and inexpensive production of the upper part and lower part from monolithic (one-piece) blanks, into which the multiplicity of wells can be introduced, in which case the elevations of the sealing beads extending around the wells can be milled out from the blank.

Surprisingly, it has been found to be particularly advantageous if the filter medium fixed between upper part and lower part can be fixed by respective pairs of the sealing means of the upper part and lower part. This ensures that the filter medium, on the permeate side thereof, does not lie across its entire surface on the side of the lower part facing toward the filter medium, which would promote undesired radial cross-diffusion of analytes.

In order to avoid cross-contamination between adjacent wells, while at the same time ensuring a high rate of flow of filtered fluid through the multi-hole plate according to the invention, it has proven useful, in a particularly preferred embodiment, if the multiplicity of wells in the upper part are cylindrical channels with a diameter d₁, and the multiplicity of wells arranged in the lower part, and communicating with these cylindrical channels, have a transition area on their side facing toward the filter medium, which transition area narrows from the diameter d₁ to a diameter d₂, and, on the side of the lower part facing away from the filter medium, the transition area is adjoined in each case by a cylindrical channel with the diameter d₂.

Optimal results in terms of avoiding cross-contamination, while at the same time ensuring a very high rate of flow of fluid through the multi-well plate, are achieved if the ratio between the aforementioned diameters d₁ and d₂ is at least 4.0, preferably at least 6.6 and particularly preferably at least 8.0.

Preferably, the upper part is connected to the lower part by screwing, gluing or latching, or by a clamp or snap-fit connection. A screw connection is particularly preferred.

According to another preferred embodiment of the invention, the upper part and/or the lower part are/is made of aluminum or plastic. In this embodiment, monolithic and apertured blanks for the lower part and upper part can be produced particularly inexpensively, and the sealing means that extend around the wells of the upper part and of the lower part can be produced by milling.

The present invention further relates to a method for characterization of a filter medium using the multi-well plate according to the invention, said method comprising the steps of:
A) pre-wetting the filter medium with a fluid,
B) inserting the filter medium into a receiving area of the lower part of the multi-well plate and connecting the upper part of the multi-well plate to the lower part, with paired fixing of the filter medium by the sealing means of the multi-well plate,
C) filtering at least one analyte through the filter medium via the communicating wells of the upper part and lower part of the multi-well plate, wherein the analyte has at least one physical and/or chemical interaction with the filter medium,
D) removing the upper part from the lower part, and
E) withdrawing the filter medium from the receiving area and analyzing the interaction that took place in step C) between filter medium and analyte.

Moreover, a use of the multi-well plate according to the invention is proposed for high-throughput analysis for characterization of membranes for separation of substances by adsorption. Uses according to the invention include automated chromatography processes for screening and optimization of polishing and capturing processes in the purification of proteins and microorganisms, the examination of interactions between amino acid compounds and the stability and behavior of membranes with respect to different substances, and simultaneous chemical reactions of one and the same filter medium with variable modifying reagents, or enzyme immobilization for various bioanalytical detection methods.

A particularly preferred use of the multi-well plate according to the invention is one in which membranes are characterized by recording a breakthrough curve for a protein.

Further features of the invention will become clear from the following description and from the attached figures in which preferred embodiments of the invention are illustrated by way of example.

### BRIEF DESCRIPTION OF THE DRAWINGS

FIG. 1 shows an exploded view of a multi-well plate according to the invention consisting of upper part, filter medium and lower part.

FIG. 2 shows a cross section through the multi-well plate according to the invention from FIG. 1, with upper part, filter medium, lower part, and pairs of sealing elements fixing the filter medium.

FIG. 3 shows staining results for the filter medium in a multi-well plate according to the invention.

FIG. 4 shows staining results for the filter medium in a multi-well plate from the prior art.

FIG. 5 shows results for phosphate detection according to Cooper in a multi-well plate according to the invention.

FIG. 6 shows a breakthrough curve for bovine serum albumin, which curve is detected using the multi-well plate according to the invention.

FIG. 7 shows the influence of the (NH₄) ₂SO₄ concentration in aqueous solution on the binding capacity of a stack of membranes of the multi-well plate according to the invention for ovalbumin, lysozyme and immunoglobulin.

FIG. 8 shows the influence of the endotoxin quantity, with which the membrane is charged at different charging volumes, on the endotoxin binding capacity of a stack of membranes of the multi-well plate according to the invention.

### DETAILED DESCRIPTION OF THE PREFERRED EMBODIMENTS

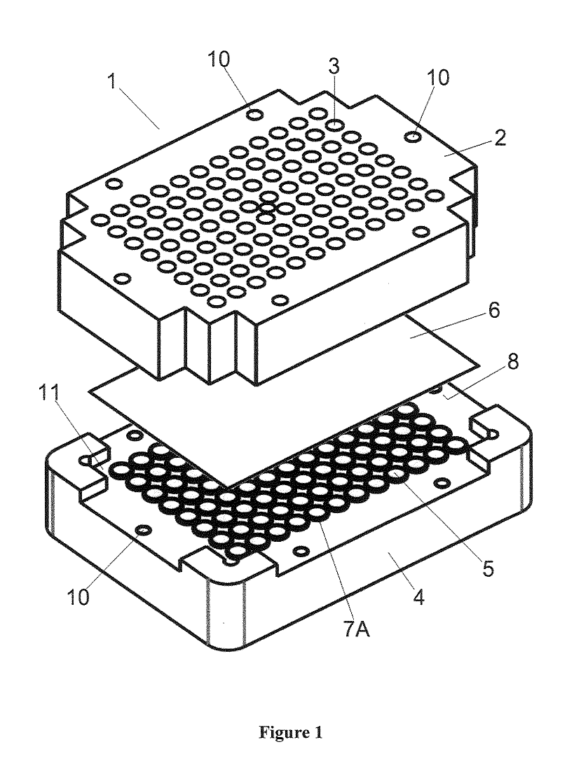

FIG. 1 shows an exploded view of a multi-well plate 1, which consists of an upper part 2 with a multiplicity of wells 3, a lower part 4 with a multiplicity of wells 5, and a filter medium 6 located between upper part 2 and lower part 4. FIG. 1 also shows sealing means 7a, which extend around the wells 5 on the side 8 of the lower part 4 facing toward the filter medium 6. The corresponding sealing means 7b on the side 9 of the upper part 2 facing toward the filter medium 6 are not shown. The sealing means 7a and 7b can be annular beads formed integrally on the upper part 2 and lower part 4 or milled out from the upper part 2 and lower part 4, or they can be 0-rings which are inserted or adhesively bonded into depressions or grooves (not shown) extending around the wells 3 and 5.

In a preferred embodiment of the invention, the upper part 2 and lower part 4 have screw thread bores 10 for the insertion of screws (not shown), with which the upper part 2 is screwed onto the lower part 4. The filter medium 6 is preferably present as a sheet and is cut to the size of the receiving area 11. The filter medium is preferably inserted into the receiving area 11 when wetted with a fluid, i.e. when moist, e.g. after complete wetting by a buffer or saline solution, and this is followed by connection of the lower part 4 to the upper part 2 to form the ready-to-use multi-well plate 1.

After a filter medium 6 that has been cut to size is inserted, the upper part 2 is screwed onto the lower part 4, and the sealing means 7a and 7b fix and seal the filter medium 6 for each well individually. Here, a number of isolated reaction spaces corresponding to the multiplicity of wells in upper part 2 and lower part 4 are tightly sealed off by the sealing means 7a and 7b with in each case a part of the filter medium 6. This tight separation of the individual reaction spaces permits a uniform wetting of the individual filter media sections enclosed by the wells.

The multi-well plate 1 according to the invention particularly preferably consists of two anodized aluminum plates, each with 96 communicating wells in the upper part 2 and in the lower part 4 for filling the wells.

The underside of the lower part 4 can be connected to a fluid-collecting device (not shown) with a vacuum generator (e.g. a pump), in order to convey fluids through the individual reaction spaces and, if appropriate, to collect the individual permeate streams from each reaction space below the lower part 4 and, if appropriate, analyze them. When analysis is complete, the upper part 2 can be removed from the lower part 4 by undoing the screw connection, and the filter medium 6 can be withdrawn and replaced by a new filter medium 6. A filter medium 6 withdrawn from the multi-well plate 1 can be subjected to a further analysis in which, for example, proteins or substrates that are bound to individual filter medium areas previously delimited by the sealing means 7a and 7b can be individually re-examined on the extracted filter medium.

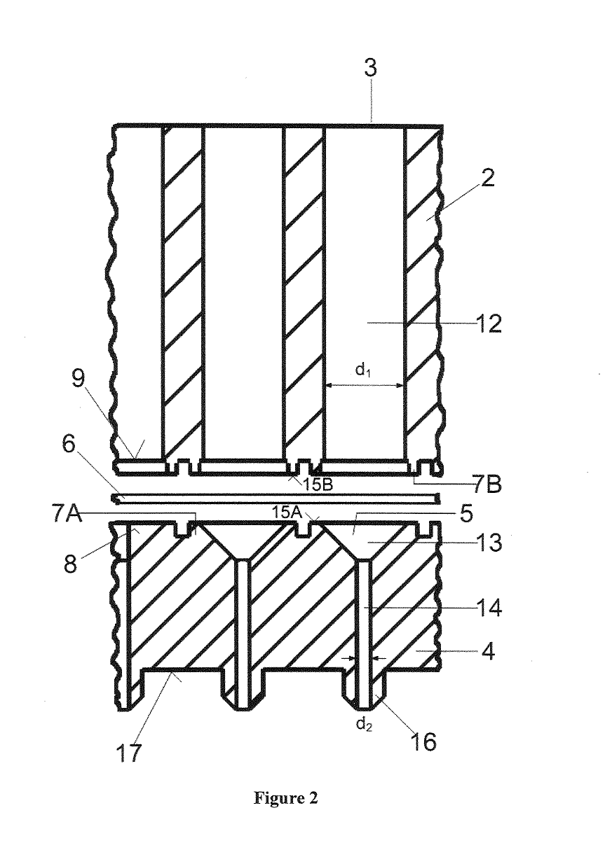

FIG. 2 shows, in an exploded view, a cross section through the multi-well plate 1 according to the invention from FIG. 1. In this embodiment of the multi-well plate according to the invention, the wells 3 in the upper part 2 form cylindrical channels 12 with a diameter d₁, while the wells 5 in the lower part have, on the side 8 facing toward the filter medium 6, a particularly preferably frustoconical transition area 13, which narrows from the diameter d₂ to the diameter d₂, and, on the side 17 facing away from the filter medium 6, the transition area 13 is adjoined by a cylindrical channel 14 with the diameter d₂. Sealing means 7b and 7a extending around the holes 3 of the upper part 2 and around the holes 5 of the lower part 4, respectively, are particularly preferably formed integrally as annular sealing beads on the upper part and lower part, e.g. in the form of O-rings, or are milled out therefrom.

Each pair of sealing means 7a and 7b ensures a sealing effect limited to the front faces 15a and 15b of the sealing means 7a and 7b, such that the effective filtration surface per well has no contact with the sealing means 7a and 7b. It is thus ensured that the filtration medium 6, on the permeate side thereof, does not lie across its whole surface area on the side 8 of the lower part 4 facing toward the filter medium 6, with the result that cross-diffusion and cross-contamination between adjacent reaction spaces are excluded.

The cylindrical channels 14 of the wells in the lower part 4 are preferably designed, in comparison with the diameter d₁ of the cylindrical channels 12 in the upper part 2, as significantly thinner capillaries with the diameter d₂, wherein the lower part of these cylindrical channels 14 leads into an opening 16, which has tapering side walls and which facilitates the dripping of the permeate into a fluid-collecting device (not shown) with a vacuum means.

When the multi-well plate 1 according to the invention is in the state ready for use, the pressure applied by the screwing operation presses the sealing means 7a and 7b firmly onto the filter medium 6 on both sides and in pairs, thereby preventing cross-contamination (e.g. with proteins) caused radially to the direction of flow by capillary forces of the filter medium 6.

### EXAMPLES

The present invention is explained in more detail below by the examples, but without in any way being limited by them.

### Example 1

Detection of the suppression of cross-contamination between adjacent reaction spaces of the multi-well plate 1 according to the invention with sealing means 7a and 7b arranged in pairs on the upper part and lower part

Dye tests are carried out to detect how the individual sections of the filter medium 6 enclosed in the reaction spaces are sealed off by the sealing means 7a and 7b in respect of binding substances. The filtration medium 6 consists of three sheets of a microporous cellulose membrane (surface area per sheet: 86 cm², thickness of each sheet 250 μm) with quaternary ammonium groups as anion-exchanging groups (Sartobind® Q Membrane from Sartorius Stedim Biotech GmbH) and, after wetting by RO (reverse osmosis) water, is placed in the receiving area 11 of the lower part 4. After it has been screwed together with the upper part 2, the lower part 4 is positioned on a vacuum-generating suction device. The reaction spaces of the assembled multi-well plate 1 are filled, via the upper part 2, with aqueous solutions (2 ml per well) of the blue, black and red dyes Methylene blue, Brilliant black and Ponceau S at concentration 0.05 g/l. A pump then generates a vacuum of 350 mbar, the dye solutions flow through the filter medium 6 and are collected in separate depressions, assigned to the individual wells, in the collection plates of the suction device. If the binding capacity of the membrane for dye is not exceeded, only the non-binding Methylene blue or Brilliant black and Ponceau S flows through the membranes of the filter medium 6 and can be found in the permeate stream.

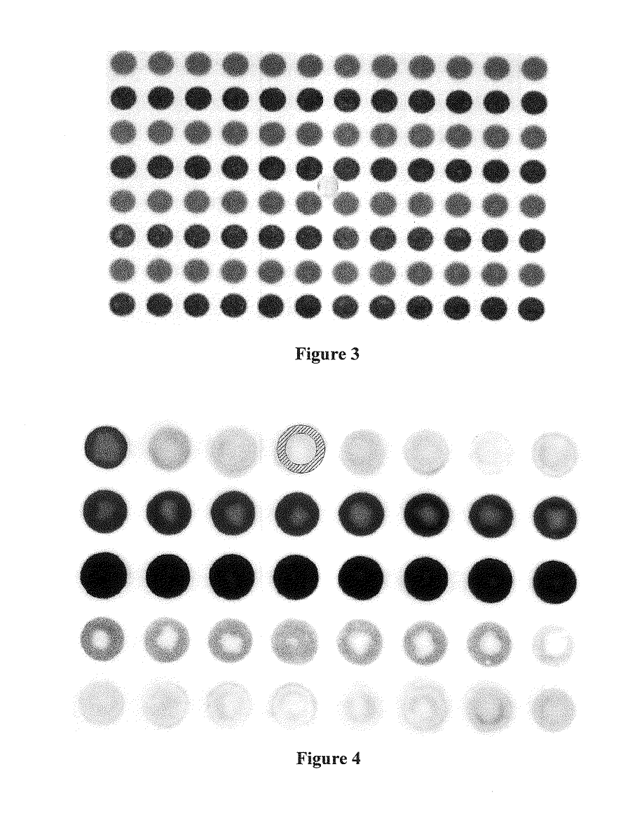

FIG. 3 shows the staining results for the aforementioned filter medium 6, in which Methylene blue, Brilliant black or Ponceau S were added alternately to the individual horizontal rows of wells 3. The staining takes place selectively in the form of homogeneously stained circles only in the membrane area that is enclosed by the annular sealing means 7a and 7b. There is no undesired radial spread of the dye molecules over the front faces 15a and 15b of the pairs of sealing means 7a and 7b and beyond the membrane surface wetted with dye solution.

### Comparison Example 1

Cross-contamination between adjacent reaction spaces in the multi-well plate known from U.S. Pat. No. 5,939,024, in which the upper part (“collimator”) has, on its underside, annular bulges (“lower rims”) around each well

The three-sheet filter medium from example 1, consisting of ion exchange membranes, is inserted into a multi-well plate as per FIGS. 2 and 3 of U.S. Pat. No. 5,939,024, in which only the upper part, on its side facing toward the filter medium, has an annular bulge around each well of the upper part, and in which the filter medium lies across its entire surface area on a plane depression in the lower part of the multi-well plate. Analogously to Example 1, the upper part as per FIGS. 2 and 3 of U.S. Pat. No. 5,939,024 is screwed onto the lower part as per FIGS. 2 and 3 of U.S. Pat. No. 5,939,024. The assembled multi-well plate is connected to the suction device from Example 1 and subjected to a defined vacuum in accordance with Example 1.

FIG. 4 shows the staining results for the aforementioned filter medium, in which 2 ml of Methylene blue solution, Ponceau S solution or Brilliant black solution were added to each well of the individual horizontal rows of wells (First row from top: Ponceau S, 0.005% by weight aqueous solution in the first well from left, Methylene blue 0.001% by weight aqueous solution in all the other wells of this row; second row from top: Ponceau S, 0.05% by weight aqueous solution; third row from top: Brilliant black, 0.05% by weight aqueous solution; fourth row from top: Brilliant black, 0.005% by weight aqueous solution; fifth row from top: Methylene blue, 0.001% by weight aqueous solution). In order to illustrate the radial cross-diffusion of the dye, the contour of the front face of the annular bulge (“lower rim”) on the underside of the upper part is indicated in the fourth well from left in the first row of the multi-well plate in FIG. 4.

In these staining results on multi-sheet membrane adsorber stacks, the device according to this comparison example shows that sufficient sealing cannot be achieved, and that there is a radial cross-diffusion of the dye over the front faces of the annular bulges on the upper part and thus beyond the limits of the individual well. In addition, the staining results from the second and fourth rows from the top show that the dye is not distributed homogeneously, as in Example 1, across the effective filter surface in the reaction space, but is instead gathered more strongly toward the edges of the well than in the middle of the well. With the annular bulges on the upper part alone, it is not possible to achieve sufficient sealing in filter media consisting of several sheets of a membrane adsorber.

### Example 2

Detection of the exclusion of cross-contamination between adjacent reaction spaces by means of the phosphate test according to Cooper

A multi-well plate 1 according to the invention, as shown in FIGS. 1 and 2, is preferably used for applications in the field of high-throughput screening, in which different charging conditions in respect of the buffer composition occur in the individual wells. A precondition for this is that the salts contained in the buffer solutions also cannot pass from one well to an adjacent well by radial cross-diffusion. To detect that inorganic salt solutions in the wells also do not cause cross-contamination, the multi-well plate 1 is equipped with three sheets of an ion exchange membrane from Example 1, pre-wetted with RO water, and is then wetted column by column with 0.5 ml phosphate solution NaH₂PO₄ at a concentration of 0.16 g/l per well. All other wells are filled with distilled water. After 15 minutes the liquids are conveyed by a pressure gradient into a collecting device. The phosphate content of the liquids in the individual collecting containers of the collecting device is determined according to Cooper (Cooper, “The Tools of Biochemistry”, Wiley-Interscience, 1977). This detection after Cooper is a highly sensitive detection method which allows phosphate ions to be detected even at a boundary concentration of 2 μg/ml by intensive blue-black staining.

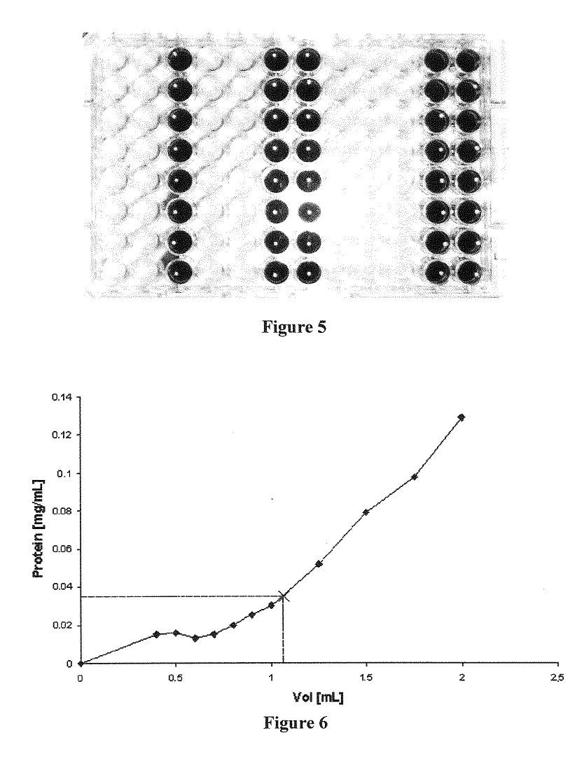

FIG. 5 shows the results of the phosphate detection according to Cooper for the charging of the 3rd, 6th, 7th, 11th and 12th columns of a multi-well plate 1 according to the invention with phosphate solution (charge 0.5 ml per well with 0.16 g/l NaH₂PO₄). The blue-black stain occurs only in the wells charged with phosphate solution, not in adjacent wells, which are flushed exclusively with distilled water.

### Example 3

Recording breakthrough curves in the high throughput method with the multi-well plate according to the invention

Breakthrough curves of membranes are generally plotted by the continuous charging of filtration units. They show whether and to what extent a membrane is able to bind or hold back one or more substances. Depending on the surface area of the membrane used, the most expensive substances often have to be applied in a large quantity. Therefore, for economic reasons, simultaneous multiple tests cannot be carried out.

The multi-well plate according to the invention allows a breakthrough curve to be recorded with minimal use of analytes. A protein solution is introduced into individual wells at a constant concentration, but in different volumes. When the charge concentration is known, the breakthrough can be recorded and assessed by analysis of the permeate. For technical purposes, it is often sufficient to detect when a breakthrough of at most 10% of the protein in the eluate is achieved.

There is an excellent correlation between breakthrough curves obtained using the multi-well plate according to the invention and breakthrough curves that were obtained conventionally by means of the aforementioned continuous charging of one and the same filtration device with rising charge concentrations.

The breakthrough capacity of a filter medium consisting of three sheets of an ion exchange membrane from Example 1, pre-wetted with RO water, in a multi-well plate 1 according to the invention with 12 wells is determined for bovine serum albumin. The 12 wells were charged with 12 different volumes of starting solution (Tris/HCl, pH 7.4) at a concentration of 0.35 g/l bovine serum albumin (in each case 6 parallel samples). The concentration of bovine serum albumin in the permeate was then determined. The evaluation shows that, with 1.1 ml of solution added to a well, more than 10% of the starting quantity of protein has broken through into the permeate. In the case of a filter medium with three sheets of membrane per well of surface area 0.7 cm², the dynamic binding capacity at 10% breakthrough is 0.54 mg/cm².

FIG. 6 shows a breakthrough curve for bovine serum albumin, this curve having been recorded using a multi-well plate according to the invention with 12 wells.

### Example 4

Examination of the influence of the salt concentration on the protein binding capacity of membrane adsorbers in the high throughput method using a multi-well plate 1 according to the invention with 96 wells

For industrial-scale processes, it is necessary to optimize process conditions for membrane adsorbers even on a small scale. Since protein interactions and buffer compositions are very complex, many attempts are often needed to find improved conditions or even suitable membranes for these processes. In the high throughput method, many process parameters can be checked simultaneously. For a filtration medium which is pre-wetted with RO water, and which consists of three sheets (surface area per sheet: cm²; thickness per sheet: 250 μm) of a crosslinked cellulose membrane containing phenyl amine ligands (Sartobind® Phenyl), it is possible, by means of a multi-well plate 1 according to the invention with 96 wells, to check 96 different conditions. In this example, 24 different conditions are tested in each case on four parallel samples. In this way, the influence of salts, for example diammonium salt ((NH₄)₂SO₄), on the binding of model proteins (here: ovalbumin, lysozyme and immunoglobulin) can be determined on a filter medium with a plurality of sheets of membrane adsorbers. For this purpose, the concentration of protein in the flow through the individual wells is determined. The more protein that can be detected in the permeate, the poorer is the binding of the protein to the membrane.

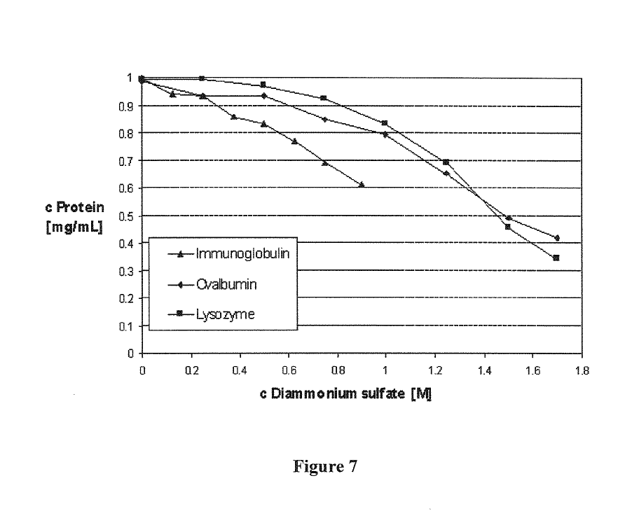

FIG. 7 shows the influence of the (NH₄)₂SO₄ concentration in aqueous solution (0.1 M sodium phosphate buffer (NaPi), pH 7; 8 concentrations of (NH₄)₂SO₄ up to a maximum of 1.7 M, charge of the respective protein per well 0.8 mg/cm², concentration in each case 1 mg/ml) on the binding capacity of the three-sheet membrane stack for ovalbumin, lysozyme and immunoglobulin. The maximum concentration of diammonium sulfate in the buffer is limited by the incipient precipitation of the respective protein.

### Example 5

Investigations concerning the influence of the endotoxin concentration and of the endotoxin charge volume on the endotoxin binding capacity of membrane adsorbers in the high throughput method using a multi-well plate 1 according to the invention with 96 wells

Endotoxins are products of decomposition of bacteria and are biologically active even at very low concentrations. They occur in different concentrations everywhere in the environment. If endotoxins enter the blood stream of living beings, they can cause adverse immune reactions. Endotoxins are lipopolysaccharides that can be inactivated only at 200° C.

In biotechnical production processes, it may be necessary, depending on the use of the end products, to remove these endotoxins in different process steps. Adsorptive membranes can also be used for endotoxin depletion in the upstream or downstream area.

By using the multi-well plate 1 according to the invention with 96 wells, the endotoxin removal can be investigated for adsorptive filter materials. It is advantageous that the multi-well plate, when the upper part 2 and lower part 4 are made of aluminum, can be reliably depyrogenated at high temperatures (>200° C.) and can thus be used more than once for the investigation of various filter materials.

In this example, endotoxin solutions (endotoxin “BIO Whittaker E. coli 05” B55 Batch 3L2770; buffer 20 mM Tris (ph 7.5), 150 mm NaCl) of various concentrations (10-1,000,000 EU (endotoxin units)/ml) at different volumes (0.25, 0.5 and 1 ml) are filtered through the multi-well plate 1 (filter medium 6, pre-wetted with RO water: Three sheets of a microporous cellulose membrane with polyallyl amine ligand, produced according to Example 21 of WO 2009/127285 A1 with a surface area per sheet of 86 cm² and a thickness per sheet of 290 μm, membrane surface area per well 1 cm²). The concentration of endotoxin in the flow-through of the individual wells is determined.

The endotoxin detection is carried out according to the “gel clot” method with the “LAL” test (limulus amoebocyte lysate). The lysate (limulus amoebocyte lysate, source: Charles River Endosafe, USA, Endosafe KTA, US License No. 1197) obtained from the horseshoe crab reacts specifically to endotoxins. The liquid and colorless lysate coagulates with endotoxin to form a firm milky gel (“gel clot”). The detection limit of the lysate used lies at 0.06 EU/ml endotoxin. This detection limit corresponds to an endotoxin concentration of 6 pg/ml. The more endotoxin that can be detected in the permeate, the poorer is the binding of the endotoxin to the membrane.

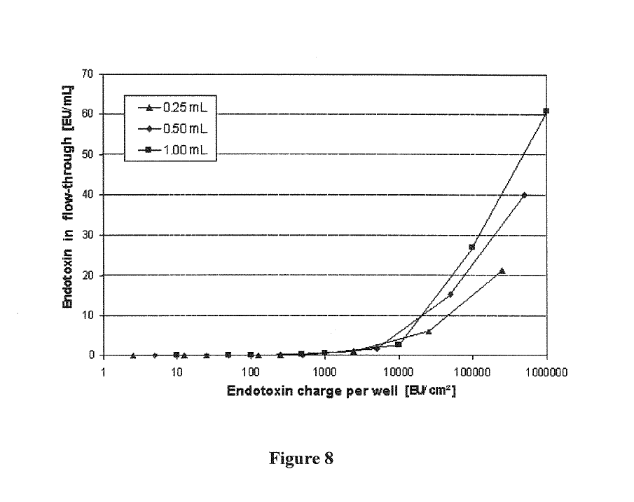

FIG. 8 shows the influence that the endotoxin quantity on the membrane has, at different charge volumes (0.25 ml, 0.50 ml and 1.00 ml), on the binding capacity of the three-sheet membrane stack for endotoxins.

1. A multi-well plate (1) comprising:
an upper part (2) with a multiplicity of wells (3),
a lower part (4) with a multiplicity of wells (5) that communicate with the wells (3) of the upper part (2), and
at least one filter medium (6) that can be fixed between the upper part (2) and the lower part (4),
characterized in that
the upper part (2) and the lower part (4), on the sides (8, 9) thereof facing toward the filter medium (6), have sealing means (7a, 7b) extending around the wells (3, 5), and
the filter medium (6) can be fixed along the upper side and lower side thereof in each case by pairs of the sealing means (7b) of the upper part (2) and the sealing means (7a) of the lower part (4).

2. The multi-well plate (1) of claim 1, characterized in that the filter medium (6) comprises at least one membrane sheet.

3. The multi-well plate (1) of claim 2, characterized in that the filter medium (6) comprises a plurality of membrane sheets stacked one on top of another.

4. The multi-well plate (1) of claim 2, characterized in that the membrane sheet is a microporous membrane to which ligands are bound.

5. The multi-well plate (1) of claim 1, characterized in that the filter medium (6) is wetted with a fluid.

6. The multi-well plate (1) of claim 1, characterized in that the sealing means (7a, 7b) are annular sealing beads formed integrally on the sides (8, 9) of the lower part (4) and upper part (2) facing toward the filter medium (6).

7. The multi-well plate (1) of claim 1, characterized in that the sealing means (7a, 7b) are annular sealing beads that are milled out on sides (8, 9) of the lower part (4) and upper part (2) facing toward the filter medium (6).

8. The multi-well plate (1) of claim 1, characterized in that the multiplicity of wells (3) in the upper part (2) are cylindrical channels (12) with a diameter dᵢ, and the multiplicity of wells (5) arranged in the lower part (4) and communicating with these cylindrical channels (12) have transition areas (13) on sides (8) facing toward the filter medium, each of the transition areas (13) narrows from the diameter d₁ to a diameter d₂, and, on a side (17) of the lower part (4) facing away from the filter medium (6), each of the transition areas (13) is adjoined in each case by a cylindrical channel (14) with the diameter d₂.

9. The multi-well plate (1) of claim 8, characterized in that the ratio between d₁ and d₂ is at least 4.0.

10. The multi-well plate (1) of claim 1, characterized in that the upper part (2) can be connected to the lower part (4) by screwing, gluing or latching, or by a clamp or snap-fit connection.

11. The multi-well plate (1) of claim 1, characterized in that at least one of the upper part and the lower part (2, 4) is made of aluminum or plastic.

12. The method of claim 14 for high-throughput analysis, characterized in that the high-throughput analysis is a characterization of filter media for separation of substances by adsorption.

13. The method of claim 12, characterized in that
at least one membrane is used as filter medium, and the membrane is characterized by recording a breakthrough curve for a protein.

14. A method for characterization of a filter medium using a multi-well plate (1) that has an upper part (2) with a multiplicity of wells (3), a lower part (4) with a multiplicity of wells (5) that communicate with the wells (3) of the upper part (2), and at least one filter medium (6) that can be fixed between the upper part (2) and the lower part (4), the upper part (2) and the lower part (4), on the sides (8, 9) thereof facing toward the filter medium (6), have sealing means (7a, 7b) extending around the wells (3, 5), and the filter medium (6) can be fixed along the upper side and lower side thereof in each case by pairs of the sealing means 17b) of the upper part (2) and the sealing means (7a) of the lower part (4), said method comprising the steps of:
A) pre-wetting the filter medium (6) with a fluid,
B) inserting the filter medium (6) into a receiving area (11) of the lower part (4) and connecting the upper part (2) to the lower part (4) with paired fixing of the filter medium (6) by the sealing means (7a, 7b),
C) filtering at least one analyte through the filter medium (6) via the communicating wells (3) and (5), wherein the analyte has at least one physical and/or chemical interaction with the filter medium (6),
D) removing the upper part (2) from the lower part (4), and
E) withdrawing the filter medium (6) from the receiving area (11) and analyzing the interaction that took place in step C) between filter medium (6) and analyte.
